(19) United States
(12) Patent Application Publication
(10) Pub. No.: US 2017/0283850 A1
(43) Pub. Date: Oct. 5, 2017

(54) METHODS OF DETECTING ADENOSINE DEAMINASE DEFICIENCY

(71) Applicant: CHILDREN'S HOSPITAL OF EASTERN ONTARIO, Ottawa (CA)

(72) Inventors: Osama ALDIRBASHI, Manotick (CA); Pranesh CHAKRABORTY, Ottawa (CA)

(21) Appl. No.: 15/513,924

(22) PCT Filed: Sep. 25, 2015
(86) PCT No.: PCT/CA2015/050963

§ 371 Date: Mar. 23, 2017

Related U.S. Application Data
(60) Provisional application No. 62/055,302, filed on Sep. 25, 2014.

(30) Foreign Application Priority Data
Sep. 26, 2014 (GB) 1417082.3

(51) Int. Cl. C12Q 1/34

(52) U.S. Cl. CPC C12Q 1/34; C12Y 305/04004; G01N 2333/978; G01N 2800/24; G01N 2458/15; G01N 2560/00

(57) ABSTRACT

Disclosed are new approaches to detecting adenosine deaminase (ADA) deficiency. There is provided a method of determining ADA activity, comprising: dividing a sample obtained from blood into two portions, adding an ADA inhibitor to one portion, measuring levels of ADA activity in both portions, and determining the ADA activity. Also provided is a method of measuring ADA substrate, comprising: measuring an ADA substrate in a sample obtained from blood of subject, and comparing this to at least one control sample obtained from blood and comprising an ADA inhibitor, and a known quantity of the ADA substrate. Multiplexed methods of measuring ADA enzymatic activity along with other metabolic markers are also provided. The methods are particularly useful for the analysis of samples obtained from dried blood spots (DBSs), and may be incorporated into existing newborn screening programs. Associated diagnostic methods, control samples, and apparatuses are also disclosed.

### FIELD

The present disclosure relates generally to metabolic screening. More particularly, the present disclosure relates to measuring adenosine deaminase deficiency.

### BACKGROUND

Newborn screening for severe combined immunodeficiency (SCID) aims at identifying affected newborns before the appearance of symptoms. Adenosine deaminase (ADA) deficiency is a rare autosomal recessive disorder of the purine salvage pathway characterized by accumulation of adenosine (Ado), deoxyadenosine (dAdo) and deoxyadenosine triphosphate (dATP). Elevations of Ado, dAdo and dATP as occurring in ADA deficiency cause systemic metabolic toxicity, which impairs the immune system and results in several non-immune abnormalities affecting hepatic, renal and neurological systems. ADA patients usually present in infancy with SCID as a result of a defective immune system [1-4]. SCID, which is characterized by impairment of cell-mediated and humoral immunity, encompasses a heterogeneous group of rare disorders and represents the severe end of the combined immunodeficiency spectrum. [1-3,5]. In ADA-SCID, unlike other causes of SCID, the cytotoxic effect of accumulating ADA substrates affects various lymphocyte subtypes and leads to T-cell, B-cell and natural killer cell lymphopenia [6]. While the overall prevalence of SCID is 1:50,000-1:100,000 live births, ADA-SCID is the second most prevalent form of SCID, accounting for 20% of cases [6-10]. Infants born with SCID appear normal at birth, however shortly after maternal antibodies decline, they are at a significant risk of life-threatening infections often leading to death [1]. The mainstay treatment for SCID in general, and ADA-SCID in particular, is hematopoietic stem cell transplantation (HSCT). ADA-SCID may also be treated with other therapeutic modalities, including enzyme replacement and gene therapy [7]. A favourable outcome is anticipated should treatment start before symptoms appear, with a higher survival rate observed in those who received transplants at or before 3.5 months of age [8].

Since 2008, a growing number of newborn screening programs have successfully implemented population-based screening for SCID [9-12]. The addition of this disorder as a primary target at Newborn Screening Ontario (NSO) was recently approved by the Ministry of Health and Long Term Care, making Ontario the first Canadian province to offer this test. In a newborn screening laboratory setting, quantitative analysis of the T-cell receptor excision circle (TREC) in dried blood spots (DBS) is the gold standard screening method [13]. However, TREC analysis alone is insufficient to determine the exact cause of SCID. This is problematic since early identification and detoxification of metabolites are vital to improving outcome for infants with ADA-SCID.

To date, there is no analytical method to measure ADA activity in DBS in a newborn screening context. Further, to develop a reliable analytical method for detecting ADA deficiency, synthetic samples containing known concentrations of purines are required. However, residual ADA activity in blood, even after traditional enzyme inactivation methods, represents a significant challenge to achieving purine analysis in this matrix. This residual activity is responsible for degrading substrates spiked into blood, hence impeding the creation of appropriate control material. Neither calibration curves nor quality control material can be prepared.

Accordingly there is a need for alternate methods to screen for ADA-SCID.

### SUMMARY

It is an object of the present disclosure to obviate or mitigate at least one disadvantage of previous approaches.

In a first aspect, there is provided a method of detecting adenosine deaminase (ADA) activity in a sample, comprising: obtaining two portions of a sample obtained from blood of a subject, adding an ADA inhibitor to one of said two portions, measuring ADA activity in said two portions, and detecting whether ADA activity is present from the two measured levels.

In another aspect there is provided a method of measuring a level of an adenosine deaminase (ADA) substrate in a blood sample, comprising: measuring at least one ADA substrate in a sample obtained from blood of a subject; measuring at least one ADA substrate in a control sample obtained from blood, wherein the control sample comprises: an ADA inhibitor, and a known quantity of the at least one ADA substrate; and determining the level of the at least one ADA substrate in the sample by comparing measurements from the sample and the control sample.

In another aspect, there is provided a multiplex method of measuring adenosine deaminase (ADA) activity in a sample, comprising: obtaining first and second portions from a sample obtained from blood of a subject, adding a labelled ADA substrate to the first portion, combining the first portion and the second portion to form a mixture, measuring a level of the at least one labelled ADA substrate in the mixture to determine ADA activity, and measuring a level of at least one additional marker in the mixture.

In a further aspect, there is provided a control sample for use in measuring, calibrating, or quality assuring an adenosine deaminase (ADA) substrate level, comprising: a sample obtained from blood, and an ADA inhibitor.

In a further aspect, there is provided an apparatus configured to carry out an above-mentioned method.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 depicts structure and product ion spectra of Ado (panel A) and dAdo (panel B) obtained by ESI-MSMS analysis.

FIG. 2 depicts MS/MS spectra obtained with neonatal DBS specimens from an ADA patient (A) and a healthy newborn (B). The asterisk denotes the stable isotope internal standard (IS) peaks.

FIG. 3 depicts the distribution of ADA activity expressed as pmol/DBS of ¹³C₁₀¹⁵N₅ Ado, ¹⁵N₅ dAdo. Solid circles represent ADA patients (n=4) and triangles represent controls (n=200).

FIG. 4 depicts purine metabolic profiles obtained from DBS specimens of an ADA deficient newborn (panel A) and that from a normal newborn (panel B). Ado and dAdo at m/z of 268 and 252, respectively are used as markers of metabolite accumulation. Peaks at m/z of 283 and 257 represent ¹³C₁₀¹⁵N₅ Ado, ¹⁵N₅ dAdo, respectively and are used to evaluate ADA activity. The asterisk denotes the stable isotope IS used for quantification.

### DETAILED DESCRIPTION

Generally, the present disclosure provides new approaches to detecting adenosine deaminase deficiency.

Measuring ADA Enzymatic Activity

To date, there is no analytical method to measure ADA activity in blood collected on blood collection paper in a newborn screening context. ADA substrate detection is impeded by residual ADA activity even after traditional enzyme inactivation methods which represents a significant challenge to achieving measurement of ADA markers.

In one aspect, there is provided a method of detecting adenosine deaminase (ADA) activity in a sample, comprising: obtaining two portions of a sample obtained from blood of a subject, adding a ADA inhibitor to one of said two portions, measuring ADA activity in said two portions, and detecting whether ADA activity is present from the two measured levels.

In one embodiment, the method may be used with a sample typically available for newborn screening. Such samples are usually collected in a way that is minimally invasive, and based on a small volume of blood.

The sample may be obtained from a dried blood spot (DBS). The sample may be extracted from a DBS. For example, the sample may be obtained by water extraction of a DBS.

In some embodiments, the method may be used with a sample comprising a small amount of starting material. For example, the sample may be from a punch from a dried blood spot (e.g., the two portions may be obtained from one punch). The punch may be less than the entirety of the DBS, such that additional DBS material remains for other samples and/or tests. For instance, the punch from the DBS may have a size of less than 18 mm². For example, the punch may be less than 17 mm², less than 16 mm², less than 15 mm², less than 14 mm², less than 13 mm², less than 12 mm², less than 11 mm², less than 10 mm², less than 9 mm², less than 8 mm², less than 7 mm², less than 6 mm², or less than 5 mm². In one particular embodiment, the punch is less than 10 mm². In another embodiment, the punch is less than 9 mm². In another embodiment, the punch is less than 8 mm². In another embodiment, the punch is about 8 mm² or less than 8 mm². In another embodiment, the punch is about 10 mm². In another embodiment, the punch is about 9 mm². In another embodiment, the punch is about 8 mm². In another embodiment, the punch is about 7 mm². The punch may be a generally circular punch.

Suitable ADA inhibitors could be selected. Suitable inhibitors include, but are not limited to erythro-9-(2-hydroxy-3-nonyl) adenine (EHNA), pentostatin, 3-Deazaadenosine, or 2-Chloro-2′-deoxyadenosine. In one embodiment, the ADA inhibitor comprises erythro-9-(2-hydroxy-3-nonyl) adenine (EHNA) or pentostatin. In one embodiment, the ADA inhibitor is EHNA.

In one embodiment, the two portions may be incubated prior to the step of measuring. First, the two portions may be incubated after extraction, for example for 5 minutes at room temperature. The two portions may be incubated for less than or about 60, 45, 30, 20, 15, 10, 9, 8, 7, 6, 5, 4, 3, 2, or 1 minute.

In one embodiment, the method comprises a step of comparing the inhibited and uninhibited portions to determine ADA activity. In one embodiment, the method comprises determining activity by measuring levels of an ADA substrate.

By ‘ADA substrate’, as used herein, is meant any natural or artificial molecule that may be processed by the ADA enzyme.

In one embodiment, the step of measuring comprises measuring the levels of at least one ADA substrate added to each of the two portions prior to the step of measuring. The two portions may be incubated with the ADA substrate prior to the step of measuring, for example for 30 minutes at 37° C. The purpose of the incubation is to allow ADA enzyme to react with the substrate, and suitable conditions could be readily selected for the incubation. The at least one ADA substrate may be at least one labelled ADA substrate.

Suitable ‘labels’ can be selected depending on the specific application and detection steps employed.

For example, fluorescent labels may be used, for example if fluorescent detection will be used to measure the level of the at least one ADA substrate. Any of a number of fluorescent moieties could be used. For example, the label may be one that is only detectable prior to or after enzymatic activity, or may undergo a change in fluorescence associated with enzymatic activity.

The labelled ADA substrate may also be an isotope-labelled ADA substrate. The isotope is preferably a stable isotope. In one embodiment, said stable isotope-labelled ADA substrate comprises ¹³C₁₀, ¹⁵N₅ adenosine and/or ¹⁵N₅ deoxyadenosine.

In some embodiments, the method has the advantage of being based on direct measurement of an ADA substrate.

It will be appreciated that a sample obtained from a subject having ADA deficiency will have low to no detectable enzyme activity, and that the difference between the measured levels of labelled ADA substrate following incubation will therefore be small to non-existent. In contrast, a subject with normal ADA activity would exhibit a difference between the levels measured in the inhibited portion vs. the non-inhibited portion.

In one embodiment, the step of measuring takes place after ADA activity has been stopped. Any compatible reagent or condition that inhibits or kills ADA activity may be used. The reaction may be stopped, for example, by adding acetonitrile.

In one embodiment, the step of measuring comprises measuring an internal standard. The internal standard may be added at the same time that ADA activity is stopped. Stopping the reaction prevents the internal standard from being consumed. The internal standard may be a labelled ADA substrate or analogue. As above, different types of labels could be used, depending on the technology. The internal standard may be another isotopically labelled ADA substrate or analogue. The internal standard may be a stable isotope labelled ADA substrate or analogue thereof. The internal standard may be distinct from the at least one ADA substrate. By “distinct” is meant that the internal standard can be distinguished or detected separately from the at least one ADA substrate mentioned above. For example, the internal standard may be ¹³C₁₀ adenosine.

In one embodiment, the method further comprises quantifying ADA activity using the internal standard. The internal standard can be added in a known amount, and comparison of the measurements of the at least one ADA substrate to the internal standard can provide an indication of how much of the substrate is present, thereby permitted ADA activity to be quantified.

In one embodiment, said step of measuring is carried out by mass spectrometry. For example, determining ADA activity in DBS may be achieved by measuring the consumption of stable isotope labelled purines (¹³C₁₀, ¹⁵N₅ adenosine and/or ¹⁵N₅ deoxyadenosine) by SIR-MS/MS using another stable isotope (¹³C₁₀ adenosine) as internal standard. This method is based on measuring the difference or ratio of ¹³C₁₀, ¹⁵N₅ adenosine and ¹⁵N₅ deoxyadenosine in samples with and without EHNA treatment.

In a further aspect, there is provided a method of screening for subjects with ADA deficiency, comprising: performing the above method of determining ADA activity, and determining that a subject has ADA deficiency if the ADA activity is below a threshold.

By “threshold” is meant a value selected to discriminate between subjects with and without ADA-SCID. The threshold may be selected according to requirements, e.g. to identify subjects having a disease, a particular increased risk thereof, or to achieve a specific sensitivity and/or specificity parameters.

Some patients with ADA deficiency may have ADA-SCID, though the clinical spectrum of ADA deficiency may be broader. In some embodiments, the method can be used to screen for ADA-SCID itself. In some embodiments, the method may be used to screen for other clinical outcomes of ADA deficiency.

The above method could be used as a first or second tier test.

In one embodiment, the above method of screening for subjects could be used as a second tier test. Second-tier testing whereby a more specific marker is measured in an original sample is an efficient way to improve the screening specificity [14-15].

In a further aspect, there is provided a method of determining the effectiveness of a treatment of adenosine deaminase deficiency, comprising: performing the above method of determining ADA activity, with a sample obtained from a subject prior to treatment to obtain a first ADA activity, and performing the same method with a sample obtained from the subject after treatment to obtain a subsequent ADA activity, and determining the effectiveness of the treatment based on the first and subsequent activities.

For example, an increase in the ADA activity in the sample obtained after treatment, as compared to the sample obtained before treatment, would be indicative of treatment efficacy. No significant change in the ADA activity would indicate that the treatment was not effective, and a decrease in ADA activity could indicate that treatment had a negative impact.

In a further aspect, there is provided a method of measuring a level of an adenosine deaminase (ADA) activity in a sample, comprising: performing the above method of determining ADA activity with a sample obtained from a subject, performing the above method of determining ADA activity with at least one control sample, and determining the level of the ADA substrate in the sample.

The step of performing the method with a control sample may be carried out for the purposes of quality assurance and/or quality control.

In one embodiment, the sample and the at least one control sample are from dried blood spots (DBSs). The sample and the at least one control sample may be obtained by water extraction of DBSs.

In one embodiment, the at least one control sample is from a healthy individual. For the purposes of testing for ADA deficiency, a healthy individual may be considered to be any person having normal levels of ADA enzymatic activity.

In one embodiment, the at least one control sample comprises two control samples, wherein an ADA inhibitor is added to one of the two control samples prior to carrying out the method. In one embodiment, the ADA inhibitor is added to one of the two control samples prior to preparing DBSs from the at least two control samples. These control DBSs may be subsequently processed in parallel to the sample obtained from a subject.

The above method involving controls may also be used to screen for subjects with ADA-SCID or to determine the efficacy of treatment thereof.

In one embodiment, there is provided a method of screening for subjects with adenosine deaminase deficiency, comprising: performing the above method, and determining that a subject has ADA deficiency if the ADA activity level is below a threshold.

In another embodiment, there is provided a method of determining the effectiveness of a treatment of adenosine deaminase deficiency, comprising: performing the above method with a sample obtained from a subject prior to treatment to obtain a first ADA activity level, performing the above method with a sample obtained from the subject after treatment to obtain a subsequent ADA activity level, and determining the effectiveness of the treatment based on the first and subsequent levels.

In some embodiments, the above-described methods may be performed in less than 5 hours, 4 hours, 3 hours, or 2.5 hours. In one embodiment, the method may be performed in 2.5 hours or less.

In one embodiment, the above methods may be applied in a newborn screening method. In one embodiment, the method may be performed using a plurality of newborn screening samples. The samples may be tested simultaneously, e.g., in parallel. The method may involve screening more than 10, 25, 50, 75, or 100 samples. The method may be adapted to samples in a standard 96-well plate format. The method may be adapted to samples in a standard 384-well plate format. The newborn screening samples may be DBSs. The method may comprise measuring a plurality of newborn screening markers for each the samples.

Measuring ADA Substrate

As mentioned, residual ADA activity in DBS even after traditional enzyme inactivation methods represents a significant challenge to achieving purine analysis. This residual activity is responsible for degrading substrates spiked into blood, hence impeding the creation of appropriate control material.

In another aspect, there is provided a method of measuring a level of an adenosine deaminase (ADA) substrate in a blood sample, comprising: measuring at least one ADA substrate in a sample obtained from blood of a subject, measuring at least one ADA substrate in a control sample obtained from blood, wherein the control sample comprises: an ADA inhibitor, and a known quantity of the at least one ADA substrate, and determining the level of the at least one ADA substrate in the sample by comparing measurements from the sample and the control sample.

A suitable “control sample” could be readily prepared corresponding to the nature of the “blood sample”. By “known quantity” is meant an amount that is known to a user.

In one embodiment, the at least one ADA substrate is an endogenous ADA substrate. By ‘endogenous’ is meant a molecule that is present in the sample, as opposed to one that is added to it.

In one embodiment, the method may be used with a sample typically available for newborn screening. Such samples are usually collected in a way that is minimally invasive, and based on a small volume of blood.

In one embodiment, the sample and the control sample are obtained from DBSs. For example, the sample and the control sample may be obtained by extraction of DBSs using a mixture of water and methanol. For example, 70% methanol may be used.

In some embodiments, the method may be used with a sample comprising a small amount of starting material. For example, the sample and/or the control sample may each be a punch from a respective dried blood spot. Each punch may be less than the entirety of the DBS, such that additional DBS material remains for other samples and/or tests. For instance, the punch from the DBS may have a size of less than 18 mm². For example, the punch may be less than 17 mm², less than 16 mm², less than 15 mm², less than 14 mm², less than 13 mm², less than 12 mm², less than 11 mm², less than 10 mm², less than 9 mm², less than 8 mm², less than 7 mm², less than 6 mm², or less than 5 mm². In one particular embodiment, the punch is less than 10 mm². In another embodiment, the punch is less than 9 mm². In another embodiment, the punch is less than 8 mm². In another embodiment, the punch is about 8 mm² or less than 8 mm². In another embodiment, the punch is about 10 mm². In another embodiment, the punch is about 9 mm². In another embodiment, the punch is about 8 mm². In another embodiment, the punch is about 7 mm². The punch may be a generally circular punch.

Suitable ADA inhibitors could be selected. Such inhibitors include, but are not limited to erythro-9-(2-hydroxy-3-nonyl) adenine (EHNA), pentostatin, 3-Deazaadenosine, or 2-Chloro-2′-deoxyadenosine. In one embodiment, the ADA inhibitor comprises erythro-9-(2-hydroxy-3-nonyl) adenine (EHNA) or pentostatin. In one particular embodiment, the ADA inhibitor is EHNA.

In some embodiments, the method has the advantage of being based on direct measurement of an ADA substrate.

In one embodiment, the at least one ADA substrate comprises adenosine (Ado), and/or deoxyadenosine (dAdo).

In one embodiment, the step of determining further comprises measuring an internal standard. The internal standard may be added concurrently with the extraction solvent. The internal standard may be a labelled ADA substrate or analogue thereof, and may be one that is distinct from the at least one ADA substrate. As in the previous section, different types of labels could be used. The label could be a fluorescent label. The label may also be an isotope label, such as a stable isotope label. For example, the internal standard may comprise ¹³C₁₀ adenosine.

In some embodiments, the method further comprises quantifying the at least one ADA substrate using the internal standard.

In one embodiment, said steps of measuring are carried out by mass spectrometry. For example, measuring ADA metabolite (adenosine and deoxyadenosine) can be achieved by tandem mass spectrometry (MS/MS) in the selected reaction monitoring (SRM) mode. The SRM is capable of including guanosine, deoxyguanosine, inosine, deoxyinosine, xanthine and hypoxanthine in the same measurement.

In a further aspect, there is provided a method of screening for subjects with ADA deficiency, comprising: performing the above method of measuring a level of ADA substrate, and determining that a subject has ADA deficiency if the ADA substrate level exceeds a threshold.

Some patients with ADA deficiency may have ADA-SCID, though the clinical spectrum of ADA deficiency may be broader. In some embodiments, the method can be used to screen for ADA-SCID itself. In some embodiments, the method may be used to screen for other clinical outcomes of ADA deficiency.

In one embodiment, the method of screening for subjects could be used as a second tier test. Second-tier testing whereby a more specific marker is measured in an original sample is an efficient way to improve the screening specificity [14-15]. In a SCID screening setting, due to the inability of TREC assay in providing information about Ado and dAdo, which are present at elevated levels in patients with ADA deficiency, analysis of these compounds in DBS specimens by another method is warranted. These markers have been shown to considerably improve newborn screening for ADA-SCID by introducing an etiologic focus.

In a further aspect, there is provided a method of determining the effectiveness of a treatment of ADA deficiency, comprising: performing the above method of measuring a level of ADA metabolite with a sample obtained from a subject prior to treatment to obtain a first ADA metabolite level, performing the same method with a sample obtained from the subject after treatment to obtain a second ADA metabolite level, and determining the effectiveness of the treatment based on the first and second levels.

For example, a decrease in the amount of ADA metabolite in the sample obtained after treatment, as compared to the sample obtained before treatment, would be indicative of treatment efficacy. No significant change in the ADA metabolite would indicate that the treatment was not effective, and an increase in the amount ADA metabolite could indicate that treatment had a negative impact.

In some embodiments, the above-described methods may be performed in less than 5 hours, 4 hours, 3 hours, or 2.5 hours. In one embodiment, the method may be performed in 2.5 hours or less.

In one embodiment, the above methods may be applied in a newborn screening method. In one embodiment, the method may be performed using a plurality of newborn screening samples. The samples may be tested simultaneously, e.g., in parallel. The method may involve screening more than 10, 25, 50, 75, or 100 samples. The method may be adapted to samples in a standard 96-well plate format. The method may be adapted to samples in a standard 384-well plate format. The newborn screening samples may be DBSs. The method may comprise measuring a plurality of newborn screening markers for each the samples.

Multiplex Method

In another aspect, there is provided a multiplex method of measuring adenosine deaminase (ADA) activity in a sample, comprising: obtaining first and second portions from a sample obtained from blood of a subject, adding a labelled ADA substrate to the first portion, combining the first portion and the second portion to form a mixture, measuring a level of the at least one labelled ADA substrate in the mixture to determine ADA activity, and measuring a level of at least one additional marker in the mixture.

By ‘marker’, as used herein, is meant any biological molecule whose presence, absence, or abundance in indicative of a biological state, such as a disease. A ‘marker’ encompasses, but is not limited to, substrates and metabolites.

In one embodiment, the method may be used with a sample typically available for newborn screening. Such samples are usually collected in a way that is minimally invasive, and based on a small volume of blood.

In one embodiment, the sample may be a dried blood spot (DBS). For example, the first and second portions may be obtained from first and second punches from a DBS. The first portion (intended for measurement of enzymatic activity) may be extracted with water. The second portion (intended for measurement of an endogenous marker) may be extracted with a mixture of water and methanol. For example, 70% methanol may be used.

In some embodiments, the method may be used with a sample comprising a small amount of starting material. For example, the sample may be from one or more punch from a dried blood spot. The first and second portions may be from first and second punches (e.g. from a single DBS). Each punch may be less than the entirety of the DBS, such that additional DBS material remains for other samples and/or tests. For instance, the punch from the DBS may have a size of less than 18 mm². For example, the punch may be less than 17 mm², less than 16 mm², less than 15 mm², less than 14 mm², less than 13 mm², less than 12 mm², less than 11 mm², less than 10 mm², less than 9 mm², less than 8 mm², less than 7 mm², less than 6 mm², or less than 5 mm². In one particular embodiment, the punch is less than 10 mm². In another embodiment, the punch is less than 9 mm². In another embodiment, the punch is less than 8 mm². In another embodiment, the punch is about 8 mm² or less than 8 mm². In another embodiment, the punch is about 10 mm². In another embodiment, the punch is about 9 mm². In another embodiment, the punch is about 8 mm². In another embodiment, the punch is about 7 mm². The punch may be a generally circular punch.

In one embodiment, the first portion is obtained by water extraction.

In one embodiment, the second portion is obtained by extraction for the at least one additional screening marker.

In one embodiment, the at least one additional marker may be at least one endogenous ADA substrate. By ‘endogenous’ is meant a molecule that is present in the sample, as opposed to one that is added to it. The at least one endogenous ADA substrate may comprise, for example, adenosine (Ado), and/or deoxyadenosine (dAdo). Accordingly, in some embodiments, the multiplex method provides information about the labelled substrate and the endogenous substrate, thereby providing a more robust assessment in some embodiments.

In one embodiment, the at least one additional screening marker comprises a plurality of screening markers. The screening markers may be selected from markers linked to disease. For example, the screening markers could be selected from newborn screening markers. For example, the screening markers may be selected from the group consisting of amino acids, acylcarnitines, and succinylacetone.

As above, suitable ‘labels’ could be selected depending on the specific application and detection steps employed.

For example, fluorescent labels may be used, for example if fluorescent detection will be used to measure the level of the at least one ADA substrate. Any of a number of fluorescent moieties could be used. For example, the label may only be detectable prior to or after enzymatic activity, or may undergo a change in fluorescence associated with enzymatic activity.

In one embodiment, the labelled ADA-substrate is an isotope-labelled ADA substrate. In one embodiment, the isotope-labelled ADA substrate is a stable isotope-labelled ADA substrate. In one embodiment, the stable isotope-labelled ADA substrate comprises ¹³C₁₀, ¹⁵N₅ adenosine and/or ¹⁵N₅ deoxyadenosine.

In some embodiments, the method has the advantage of being based on direct measurement of an ADA substrate.

In one embodiment, ADA activity is stopped prior to the step of combining. ADA activity may be stopped, for example, by adding acetonitrile.

In one embodiment, the step of measuring comprises measuring an internal standard. The internal standard may be added at the same time that ADA activity is stopped. The internal standard may be a labelled ADA substrate or analogue. The internal standard may be another isotopically labelled ADA substrate or analogue distinct from the at least one labelled ADA substrate. For example, the internal standard may be ¹³C₁₀ adenosine.

In one embodiment, the steps of measuring are carried out simultaneously.

In one embodiment, said step of measuring is carried out by mass spectrometry.

In a further aspect, there is provided a method of screening for ADA deficiency, comprising: performing the above multiplex method, and determining that a subject has ADA deficiency if the ADA activity is below a threshold. In one embodiment, ADA deficiency may be identified if the endogenous substrate is above a particular threshold.

Some patients with ADA deficiency may have ADA-SCID, though the clinical spectrum of ADA deficiency may be broader. In some embodiments, the method can be used to screen for ADA-SCID itself. In some embodiments, the method may be used to screen for other clinical outcomes of ADA deficiency.

Likewise, multiplex analysis that includes an assessment of other markers could be used to screen for multiple conditions. For example, guanosine and deoxyguanosine are markers of PNP deficiency, while xanthine and hypoxanthine are markers of molybdenum cofactor deficiency. Other markers could also be used.

The above-described multiplex method may be used as a first or second-tier test. In one embodiment, the above-described multiple method is used a first-tier test. For example, it may be used in a newborn screening program. In one embodiment, a subject identified as having ADA deficiency (or a risk thereof) could be tested with a second-tier test, such as those described herein under ‘Measuring ADA Enzymatic Activity’ or ‘Measuring ADA Substrate’.

In a further aspect, there is provided a method of determining the effectiveness of a treatment ADA deficiency, comprising: performing the above method with a sample obtained from a subject prior to treatment to obtain a first measurement of ADA activity and/or substrate, performing the same method with a sample obtained from the subject after treatment to obtain a measurement of ADA activity and/or substrate, and determining the effectiveness of the treatment based on the first and second measurements.

In some embodiments, the above-described methods may be performed in less than 5 hours, 4 hours, 3 hours, or 2.5 hours. In one embodiment, the method may be performed in 2.5 hours or less.

In one embodiment, the above methods may be applied in a newborn screening method. In one embodiment, the method may be performed using a plurality of newborn screening samples. The samples may be tested simultaneously, e.g., in parallel. The method may involve screening more than 10, 25, 50, 75, or 100 samples. The method may be adapted to samples in a standard 96-well plate format. The method may be adapted to samples in a standard 384-well plate format. The newborn screening samples may be DBSs. The method may comprise measuring a plurality of newborn screening markers for each the samples.

Control Material

In another aspect, there is provided a control sample for use in measuring, calibrating, or quality assuring an adenosine deaminase (ADA) substrate level, comprising: a sample obtained from blood, and an ADA inhibitor.

In one embodiment, the control sample further comprises an ADA substrate. The ADA substrate may be a labelled ADA substrate. The labelled ADA substrate may be a fluorescent-labelled ADA substrate. The labelled ADA substrate may be an isotope labelled ADA substrate. The isotope-labelled ADA substrate may be a stable isotope-labelled ADA substrate. The stable isotope labelled ADA substrate may be ¹³C₁₀, ¹⁵N₅ adenosine or ¹⁵N₅ deoxyadenosine.

In one embodiment, the sample may be from a dried blood spot (DBS). For example, the sample may be water-extracted from a DBS.

The ADA substrate may be present in known quantity.

The control sample may be in the form of a dried blood spot (DBS).

Suitable ADA inhibitors could be selected. In one embodiment, the ADA inhibitor comprises using erythro-9-(2-hydroxy-3-nonyl) adenine (EHNA) or pentostatin. In one embodiment, the ADA inhibitor is EHNA.

The control sample(s) may be quality control samples.

Apparatus

In a further aspect, there is provided an apparatus configured to carry out the above-mentioned methods. In one embodiment, the apparatus is configured to carry out the above-described multiplex method. The apparatus may also be configured to carry out parallel analysis of multiple samples. In some embodiments, the apparatus comprises a mass spectrometry unit. In some embodiments, the apparatus comprises sample handling equipment. The apparatus may set up for person to operate. The apparatus may also comprise robotics. The apparatus may permit automated sample handling. The apparatus may be configured to process a plurality of samples in parallel.

### Example 1

Materials and Methods

Chemicals and Standard Solutions

Ado, dAdo, Gua and dGua were supplied by Sigma-Aldrich (St. Louis, Mo., USA). ¹³C₅ Ado, ¹³C₁₀ ¹⁵N₅ Ado, ¹⁵N₅ dAdo, ¹⁵N₅ Gua and ¹⁵N₅ dGua used as internal standards (IS) were purchased from Cambridge Isotope Laboratories (Andover, Mass., USA). LC-MS grade acetonitrile and LC-MS grade methanol were from Burdick's and Jackson (Muskegon, Mich., USA). LC-MS grade formic acid was purchased from Fisher Scientific (Fair Lawn, N.J., USA). Erythro-9-(2-hydroxy-3-nonyl) adenine (EHNA) was from Sigma-Aldrich (St. Louis, Mo., USA). Water was obtained by Direct-Q 5 UV-R Ultra pure water system (Millipore S.A.S. Molsheim, France). All other reagents were of analytical grade or better.

Individual solutions of purines and labeled IS at a concentration of 1.0 mg/ml were prepared by dissolving proper amounts of standard material in water for Ado, dAdo and dGua, in 50% methanol for ¹³C₅ Ado, ¹⁵N₅ dAdo and ¹⁵N₅ dGua and in ammonium hydroxide (10 mmol/L) for Gua and ¹⁵N₅ Gua. A mixture of ¹³C₅ Ado, ¹⁵N₅ dAdo, ¹⁵N₅ Gua and ¹⁵N₅ dGua at a concentration of 0.1 mmol/L was prepared in 50% methanol and was further diluted in the same solvent to produce the intermediate IS solution at 1.0 μmol/L. These solutions are stable for at least 6 months when stored at −20° C. in the dark. A daily IS solution at a concentration of 0.1 μmol/L is freshly prepared by diluting the intermediate IS solution 10 fold in 70% methanol.

Control and Patient DBS Samples

The Institutional Research Ethics Board of the Children's Hospital of Eastern Ontario (CHEO) approved this study. Anonymized, archived DBS samples from the Newborn Screening Ontario laboratory, which produced normal profiles for all screened conditions were used to determine the reference ranges of purines (n=588) and ADA activity (n=200). These samples are collected in general at 24-72 hours. Archived DBS specimens from confirmed ADA patients (n=4) were also analyzed. These samples were stored at ambient temperature under dry conditions for up to 80 months.

Sample Preparation for Purine Measurements

To a single 3.2 mm DBS disc placed in a designated well of a 96 well plate, 100 μl of daily IS solution was added and the plate was sealed with a sealing film (Platemax, Axygen Scientific). After incubation with shaking (37° C., 650 rpm) for 15 minutes, 90 μL of eluates were transferred to a 96 well Nunc plate (Thermo Scientific) and evaporated to dryness under vacuum (60° C., 45 min). The residue was reconstituted in 90 μl of 0.1% formic acid in 70% acetonitrile by shaking for 10 minutes at 27° C. Aliquots of 7.5 μl of the resultant solution were injected onto the MS/MS system.

Determination of ADA Enzyme Activity

A 3.2 mm DBS sample was punched into the designated well of a 96 multi-well filter plate (Pall Corp, Ann Arbor, Mich., USA) and eluted using 120 μl of water by shaking at 650 rpm (24° C. for 30 min). After filtration under vacuum, two 40 μl portions of the eluate were dispensed into two 2 ml microtubes (Axygen, Union City, Calif., USA) and labeled Test and Blank. To the tube labeled Test, 10 μl of 10 μmol/l of EHNA in water were added whereas water (10 μl) was added to Blank tubes. The tubes were vortexed for 10 sec and allowed to sit at room temperature for 5 min. To each tube, 50 μl 2 mmol/L ammonium acetate containing 1 μmol/L of ¹³C₁₀, ¹⁵N₅ Ado and ¹⁵N₅ dAdo were added as ADA substrates. The mixture was incubated at 37° C. with shaking (20 rpm). After 30 minutes, the enzymatic reaction was stopped by adding 400 μl of acetonitrile containing ¹³C₅ Ado (0.125 μmol/L) and the mixture was vortexed for 30 sec. After evaporation to dryness using a vaccufuge for 55 min at 60° C., the residue was reconstituted in 125 μl of water containing 0.1% formic acid and 3 μl of this mixture were injected into the MS/MS system to measure residual ¹³C₁₀, ¹⁵N₅ Ado and ¹⁵N₅ dAdo using ¹³C₅ Ado as IS. The enzyme activity expressed as pmol of residual substrate per DBS was measured by calculating the difference of ¹³C₁₀, ¹⁵N₅ Ado and ¹⁵N₅ dAdo in Test and Blank after the enzymatic reaction.

MS/MS System

Here is presented a novel MS/MS method to detect Ado, dAdo, guanosine (Gua) and deoxyguanosine (dGua), collectively referred to as purine metabolites, in DBS. This method utilizes a simple sample preparation and allows for the detection of these metabolites in a single 3.2 mm disc. A procedure to measure ADA activity in DBS using stable isotopes as substrates is also described. These methods can be applied to DBS specimens with low TREC counts as a second-tier test to provide additional information and to guide and expedite diagnostic workup and treatment.

Analysis of purines in DBS was performed on a Xevo XE MS/MS system (Micromass, Manchester, UK) coupled with Waters ACQUITY Ultra Performance LC system (Waters, Milford, Mass., USA) for solvent delivery and sample introduction. MassLynx software (V4.1) running under Microsoft Windows XP professional environment was used to control the instruments and for data acquisition.

The electrospray ionization source (ESI) was operated in the positive ion mode using a capillary and cone voltage of 3.0 kV and 29 V, respectively with a collision energy of 10 eV using argon as collision gas. Ion source and desolvation temperatures were maintained at 120 and 400° C., respectively. Scanning was in the multiple reaction monitoring (MRM) mode using transitions of mass to charge (m/z) of 268 to 136 for Ado, 273 to 136 for ¹³C₅ Ado, 283 to 136 for ¹³C₁₀, ¹⁵N₅ Ado, 252 to 136 for dAdo, 257 to 136 for ¹⁵N₅ dAdo, 284 to 152 for Gua, 289 to 157 for ¹⁵N₅ Gua, 268 to 152 for dGua and 273 to 157 for ¹⁵N₅ dGua with a dwell time of 0.03 second.

Samples were introduced to the ion source using 70% acetonitrile containing 0.1% formic acid as mobile phase. The flow rate gradient was programmed to start at 140 μl/min then dropped to 10 μl/min after 0.2 minutes. At 1.21 minutes, the flow was increased to 500 μl/min. This surge in flow at the end of data acquisition serves to clear any residual material and to decrease the background noise. Injection to injection time was set at 2.5 min.

Method Development and Validation

Extraction of purines was optimized using aqueous organic mixtures at various proportions. Extraction time was investigated using 70% methanol for various time periods. Linear ranges were determined using calibrators prepared with standard purine solutions diluted in 0.9% NaCl (Baxter, Mississauga, ON, Canada) in the range of 0.1 to 100 μmol/L. Quality control (QC) materials at physiological and pathological purine levels were prepared using whole blood with or without EHNA treatment at a concentration of 10 μmol/L. Calibrators and QCs were applied manually onto Whatman 903™ Specimen Collection Paper and allowed to dry at ambient temperature overnight. Dried calibrators and QC samples were stored at −20° C. in sealed plastic bags with a desiccant.

Within-day (n=12) and between-day (n=12) variations were evaluated by repeatedly analyzing QC materials at levels representing normal and abnormal concentrations. Coefficient of variation (CV %) was calculated according to the following equation [CV %=100×standard deviation/mean]. Analytical recovery was calculated using data obtained from DBS specimens as follow [Recovery %=100×(concentration measured)/concentration added].

Stability of purine metabolites in DBS was assessed by storing spiked samples (5 and 25 μmol/L) at various temperatures (ambient, −20° C. and 30° C.). Analysis was carried out as described over a period of 5 weeks.

ADA activity assay conditions were determined by monitoring the enzyme reaction (up to 60 min), substrate concentration (1-10 μmol/L), EHNA concentration (2.5-160 NM) and incubation temperature (30-60° C.). Infra-day (n=20) and inter-day (n=20) reproducibility of ADA activity analysis were assessed by repeatedly analyzing a normal and EHNA-treated DBS specimens.

### Example 2

Results

MS/MS Experiments

Individual solutions containing purine metabolites and IS were infused into the first quadrupole of the MS/MS. Scanning in positive ion mode ESI-MS revealed intense ions at m/z of 268, 252, 284 and 268 corresponding to [MH]⁺ of Ado, dAdo, Gua and dGua, respectively. Subsequent transmission of these ions into the collision cell, followed by scanning using the second resolving quadrupole for fragments, revealed a common fragmentation pattern corresponding to the cleavage of the glycosidic C—N bond. Intense fragments produced from Ado and dAdo were assigned to protonated adenine (m/z of 136) whereas those from Gua and dGua were assigned to protonated guanine (m/z of 152).

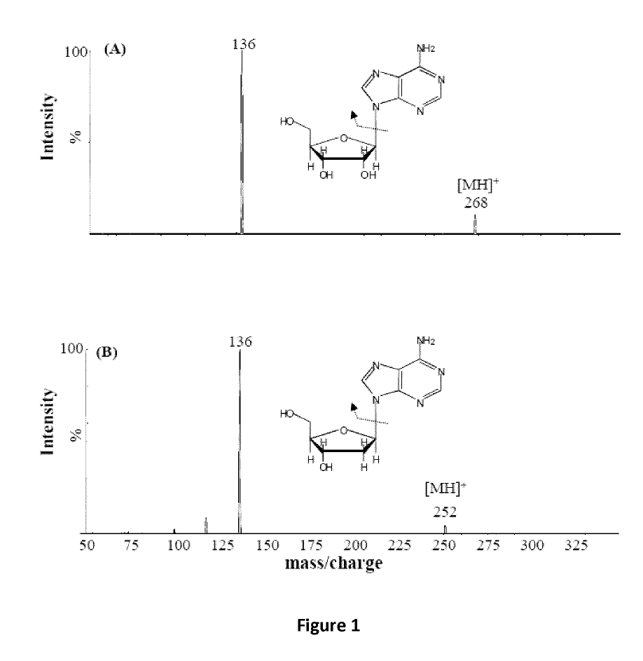

FIG. 1 shows the product ion spectra and fragmentation pattern of Ado at m/z of 268 (FIG. 1, panel A) and dAdo at m/z of 252 (FIG. 1, panel B).

Chromatographic separation was not required in this work and samples were introduced into the MS/MS using a flow injection analysis method. This was achieved using a gradient program that changes the flow rate of 70% (v/v) acetonitrile containing 0.1% formic acid between 10-500 μl/min over the course of the run to maximize the sensitivity. The use of flow surge at the end of each run reduced ion suppression and enhanced the peak shape. The analytical time between successive injections was 2.5 min.

Sample Preparation for Purine Measurements

DBS calibrators could not be prepared in this work due to residual ADA activity that persisted after traditional enzyme deactivation treatments such as freeze-thawing or heating whole blood at 45° C. for 24 hours. EHNA, a specific ADA inhibitor, was added to whole blood to prevent the deamination of Ado and dAdo to inosine and deoxyinosine, respectively. Purine metabolites were extracted from 3.2 mm dried calibrators or DBS specimens using an aqueous solution of 70% methanol (v/v) containing isotope labeled IS. This solution was added directly into a 96-well plate containing samples and incubated at 37° C. with shaking (650 rpm). The extraction yield reached its maximum at 15 minutes or more. The following experiments therefore were performed at 37° C. for 15 min. Purine metabolites were stable for at least 24 h when stored in a tightly sealed vial at 8° C.

Sample Preparation for ADA Activity Measurements in DBS

Optimum conditions for ADA activity measurements were EHNA at a concentration of 10 μM or more, substrate concentration of 1.0 μmol/L and incubation at 37° C. for 30 minutes or more.

Assay Validation

Regression analysis of analyte-to-IS peak ratios versus concentration in dried calibrators revealed linear relationships between 0.1-100 μM for all studied compound. Analysis of DBS specimens containing Ado, dAdo, Gua and dGua at 5 and 25 μmol/L stored for a period of 4.5 weeks at −20° C., 23° C. (ambient) and 30° C., revealing that these compounds are stable at the conditions described.

Within-day (n=12) and between-day (n=12) imprecisions of purine measurements were evaluated by repeated analysis of DBS QC samples.

Table 1 summarizes the imprecision expressed as coefficient of variation (%) and analytical recovery obtained using dried calibrators.

TABLE 1
Recovery, within-day and, between-day
reproducibility of Ado and dAdo in DBS

| | | Within- | | | | | |
|---|---|---|---|---|---|---|---|
| | | day | | Between-day | | | |
| | | (n = 12) | | (n = 12) | | Recovery | |
| | Concentration | Mean | CV^{b} | Mean | CV | (%) | |
| Compound | added (μM) | (μM) | (%) | (μM) | (%) | Mean | CV |
| Ado | 0^{a} | 0.67 | 9.6 | 0.55 | 12.4 | 124.8 | 12.8 |
| | 9.4 | 13.4 | 10.8 | 13.3 | 6.3 | | |
| | 18.7 | 21.1 | 7.8 | 19.6 | 3.7 | | |
| dAdo | 0^{a} | 0.12 | 48.5 | 0.13 | 34.9 | 85.8 | 23.6 |
| | 13.0 | 8.5 | 4.6 | 8.6 | 9.1 | | |
| | 31.6 | 33.5 | 7.1 | 33.6 | 3.1 | | |

^{a}DBS from a healthy individual that was not enriched with Ado and dAdo
^{b}Coefficient of variation
^{c}Recovery (%) = 100 × (concentration measured-concentration added)/concentration added

The inter-day (n=20) and intra-day (n=20) reproducibility of ADA activity analysis in DBS expressed as CV was better than 21.3%.

Analysis of Controls and Patients Samples

In this work, ADA enzyme activity is expressed as pmol of isotope labeled Ado or dAdo per DBS. These values were obtained by calculating the difference of residual ¹³C₁₀, ¹⁵N₅ Ado and ¹⁵N₅ dAdo in EHNA treated (i.e. Test) and non-EHNA treated (i.e. Blank) samples. In ADA deficient samples, the added stable isotope substrates are not consumed by ADA in either the Test and Blank samples and the difference between Test and Blank approaches zero. On the other hand, the observed difference between Test and Blank in normal samples is orders of magnitude higher than that in patients.

Table 2 Summarizes these results.

TABLE 2
Concentrations of TREC, purines, ADA activity and mutations in ADA-SCID Patients
and Controls

| | | | | | | ADA activity | | |
|---|---|---|---|---|---|---|---|---|
| | TREC | Purine concentration (μM) | | | | (pmol/DBS) | | |
| | #/μL | Ado | dAdo | Gua | dGua | Ado^{b} | dAdo^{c} | Mutations |
| Controls^{a} | 83-3316 | 0.9-3.0 | 0.1-0.4 | 0.5-7.4 | 0.2-3.5 | 0.8-1.6 | 0.4-0.7 | |
| Patient 1 | 0 | 21.9 | 40.5 | 2.7 | 4.1 | 0.04 | 0.01 | R142X/E319fsX321 |
| Patient 2 | 0 | 33.4 | 55.2 | 2.6 | 1.5 | 0 | 0 | R142X/TE319fsX321 |
| Patient 3 | 0 | 33.0 | 47.3 | 4.4 | 2.1 | 0 | 0 | R142X/E319fsX321 |
| Patient 4 | 0 | 51.3 | 33.7 | 3.0 | 1.7 | 0 | 0.01 | C153F/A329V |

^{a}The reference intervals (2.5%-97.5%) were generated using n = 588 and n = 200 for purines and ADA activity, respectively
^{b}ADA activity calculated using isotope labeled Ado as substrate. See text for details.
^{c}ADA activity calculated using isotope labeled dAdo as substrate. See text for details.

Reference intervals of purine metabolites (n=588) and ADA activity (n=200) in DBS samples from healthy newborns are shown in Table 2. Shown also are pathological levels obtained in DBS samples of patients with genetically confirmed ADA deficiency (n=4).

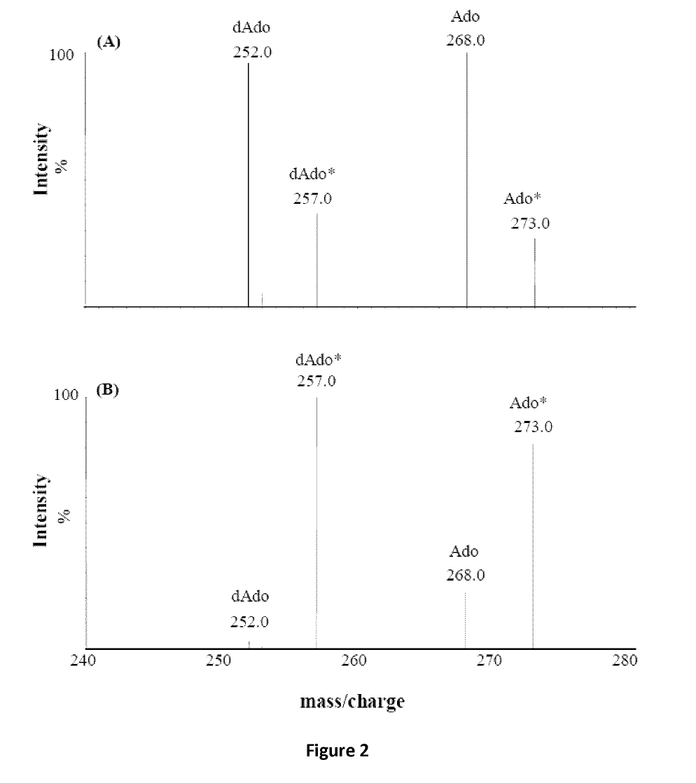

FIG. 2 shows MS/MS spectra obtained with neonatal DBS specimens from an ADA patient (FIG. 2, panel A) and a healthy newborn (FIG. 2, panel B).

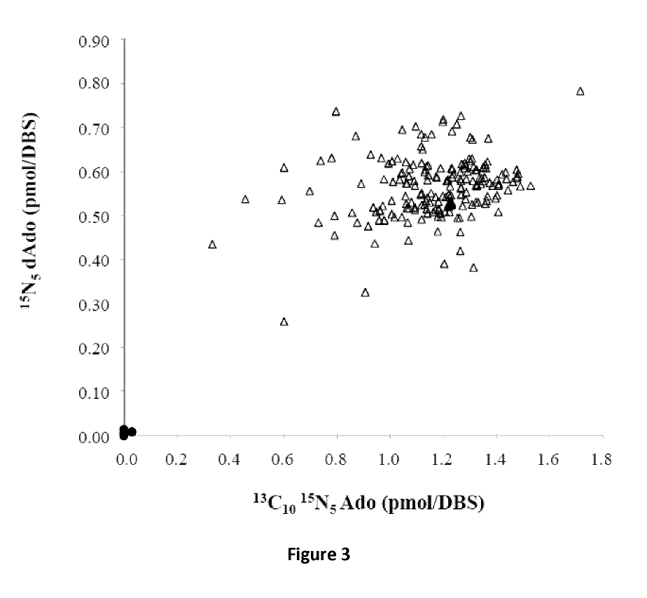

FIG. 3 depicts distribution of ADA activity expressed as pmol/DBS of 13C10 ¹⁵N₅ Ado, ¹⁵N₅ dAdo. Solid circles represent ADA patients (n=4) and triangles represent controls (n=200).

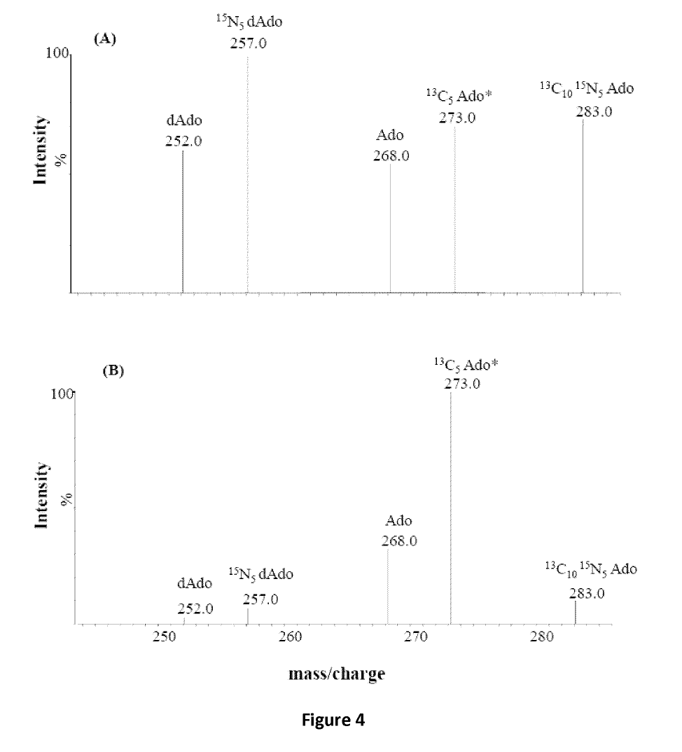

FIG. 4 depicts purine metabolic profiles obtained from DBS specimens of an ADA deficient newborn (FIG. 4, panel A) and that from a normal newborn (FIG. 4, panel B). Ado and dAdo at m/z of 268 and 252, respectively are used as markers of metabolite accumulation. Peaks at m/z of 283 and 257 represent ¹³C₁₀¹⁵N₅ Ado, ¹⁵N₅ dAdo, respectively and are used to evaluate ADA activity. The asterisk denotes the stable isotope IS used for quantification.

### Example 3

Discussion

SCID newborn screening began in the United States following the recent addition of this condition to the uniform panel as recommended by the US Department of Health and Human Services. In Canada, Ontario was the first jurisdiction to screen for SCID, which began in August, 2013. TREC analysis is the primary screening method and can be achieved by real-time PCR using neonatal DBS, the sample of choice for newborn screening. However, TREC analysis is inadequate to provide additional information regarding the etiology of SCID. This is particularly important in ADA-SCID where progressive organ damage is caused by metabolite accumulation and early treatment is associated with better outcome. ADA-SCID patients can be identified by measuring purine metabolites namely Ado and dAdo in DBS specimens. In the literature, analysis of these metabolites by MS/MS has been described, however the published method doesn't allow for preparing control samples in whole blood due to residual enzyme activity [16-18]. Further, the use of single ¹³C labeled Ado IS in the published method [17] is inappropriate as it shares the same mass transition with the natural Ado isotope. Therefore, a more reliable method that employs spiked blood controls was sought, and extended to encompass other purines such as Gua and dGua, the markers for purine nucleoside phosphorylase (PNP) deficiency [19-20]. In the early experiments, unlike Gua, and dGua, immediate loss of Ado and dAdo was observed upon adding standard purines to whole blood. A similar observation was also described by la Marca et al [17] who ascribed this to residual ADA activity and opted to use aqueous calibrators. In a clinical lab setting, control materials, should ideally be prepared in the same matrix as the target sample. Therefore, in this work pathological purine levels were achieved by spiking whole blood with EHNA and allowing this potent ADA inhibitor to restrict the enzyme activity prior to spiking the whole blood with purines. The EHNA spiked blood imitates ADA deficiency making it possible to create QC material with pathological enzyme activity and purine levels. The resultant DBS specimens were used as QC material and included in every analytical run throughout this work.

Purines are nitrogenous compounds, thus are appropriate for detection by positive ion electrospray ionization MS/MS equipment commonly used in newborn screening laboratories. The precursor ions corresponded to protonated nucleosides and the fragmentation pattern observed is common to all studied compounds and is consistent with glycosidic bond cleavage. The use of specific MRM transitions to monitor these nucleosides enabled us to maximize the sensitivity and eliminated the need for chromatographic separation. With a simple sample preparation and an MS/MS run of 2.5 min per sample, this meets the required turn-around time and integrates purines measurements as an integral part of our routine screening process for SCID.

In this work, DBS QC material was designed to cover a wide concentration range encompassing physiological and pathological Ado and dAdo levels to achieve maximum diagnostic value. The use of stable isotopes IS with five mass units greater than target analytes eliminated the interference from the naturally occurring isotopes and enhanced the quality of quantitative data obtained.

Several metabolites and enzymes can be measured in neonatal DBS specimens indicating that the dry nature of this matrix provides a favorable environment that decreases degradation. In this work, we found purines in DBS to be consistently stable for at least 4.5 weeks at temperatures ranging between −20° C. and 32° C. This is particularly important as stability during transport of DBS samples is essential to guarantee sample integrity and result validity.

As shown in Table 2, Ado and dAdo measured by the current method in DBS specimens from healthy newborns were below 3.0 and 0.4 μM, respectively. On the other hand, dAdo and a lesser extent Ado, were detected at significantly higher concentrations in SCID-ADA patients. As expected, both Gua and dGua were within normal limits in ADA patients.

ADA-SCID was confirmed by measuring ADA activity in neonatal DBS specimens. The assay used was based on measuring the consumption of ¹³C₁₀ ¹⁵N₅ Ado and ¹⁵N₅ dAdo by ADA. The enzymatic reaction product was then quantified by MS/MS using ¹³C₅ Ado as IS. Each sample was measured in duplicate with and without EHNA treatment to ensure accurate ADA measurements.

FIG. 3 shows that the method was able to clearly differentiate between ADA patients and healthy newborns, thus providing important enzymatic information from the original DBS specimen.

It has been reported that unlike TREC analysis, quantification of purine metabolites identifies newborns with late-onset ADA deficiency [18]. The potential of simultaneously measuring purine metabolites and ADA activity together with other established screening markers in a single mass spectrometric run was evaluated. This novel method involves combining the reconstituted residue of the Blank preparation described above (i.e. without EHNA treatment) with the reconstituted amino acids and acylcarnitines preparation. Quantification of these additional markers (i.e. Ado, dAdo, ¹³C₁₀, ¹⁵N₅ Ado and ¹⁵N₅ dAdo) was multiplexed into our existing screening method for amino acids and acylcarnitines in a single injection. This combination allows for our novel methodology to be used as a primary screen for ADA-SCID with sensitivity adequate of detecting ADA-SCID with no additional burden on instrument time. The use of this methodology suites the metabolic nature of ADA-SCID and complements TREC analysis by providing additional biochemical information.

FIG. 4 shows purine metabolic profiles obtained from DBS specimens of an ADA deficient newborn (FIG. 4, panel A) and that from a normal newborn (FIG. 4, panel B)

FIG. 4 also shows that multiplexed measurements of natural (endogenous) metabolites and added (labelled) metabolites is possible. It is envisaged that this method could be incorporated into existing newborn screening protocols, and that a large number of metabolites (along with the labelled ADA substrates) could be simultaneously measured.

In conclusion, purine and ADA activity measurements in neonatal DBS samples are anticipated to improve the timely identification of ADA-SCID patients with excellent sensitivity. While there was a small number of samples from patients with PNP deficiency, a disorder known to be extremely rare, the extant data suggests that Gua and dGua may be of diagnostic value.

### REFERENCES

1) van den Berghe G, Francoise Vincent M, Marie S. Disorders of purine and pyrimidine metabolism. In: Fernandes J, Saudubray J M, van den Berghe G, Walter J H, eds. Inborn metabolic diseases diagnosis and treatment. Heidelberg: Springer, 2006:433-49.
2) Nyhan W L. Pediatric endocrinology and inborn errors of metabolism. In: Sarafoglou K, Hoffmann G F, Roth K S, eds. New York: McGraw-Hill, 2009:757-86.
3) Hershfield M S, Mitchell B S. Immunodeficiency diseases caused by adenosine deaminase deficiency and purine nucleoside phosphorylase deficiency. In: Scriver D, Beaudet A, Valle D, Sly W, eds. The metabolic and molecular bases of inherited disease. New York: McGraw-Hill, 2001:2585-625.
4) Sauer A V, Brigida I, Carriglio N, Aiuti A. Autoimmune dysregulation and purine metabolism in adenosine deaminase deficiency. Front Immunol. 2012; 3:265.
5) Notarangelo L D. Primary immunodeficiencies. J Allergy Clin Immunol. 2010; 125:S182-94.
6) Kelly B T, Tam J S, Verbsky J W, Routes J M. Screening for severe combined immunodeficiency in neonate. Clin Epidemiol. 2013; 5:363-9.
7) Gaspar H B, Aiuti A, Porta F, Candotti F, Hershfield M S, Notarangelo L D. How I treat ADA deficiency. Blood. 2009; 114:3524-32.
8) Pai S Y, Logan B R, Griffith L M, Buckley R H, Parrott R E, Dvorak C C, Kapoor N, Hanson I C, Filipovich A H, Jyonouchi S, Sullivan K E, Small T N, Burroughs L, Skoda-Smith S, Haight A E, Grizzle A, Pulsipher M A, Chan K W, Fuleihan R L, Haddad E, Loechelt B, Aquino V M, Gillio A, Davis J, Knutsen A, Smith A R, Moore T B, Schroeder M L, Goldman F D, Connelly J A, Porteus M H, Xiang Q, Shearer W T, Fleisher T A, Kohn D B, Puck J M, Notarangelo L D, Cowan M J, O'Reilly R J. Transplantation outcomes for severe combined immunodeficiency, 2000-2009. N Engl J Med. 2014; 31; 371:434-46.
9) Puck J M. Neonatal Screening for Severe Combined Immunodeficiency (SCID). Curr Opin Pediatr. 2011; 23: 667-673.
10) Verbsky J W, Baker M W, Grossman W J, Hintermeyer M, Dasu T, Bonacci B, Reddy S, Margolis D, Casper J, Gries M, Desantes K, Hoffman G L, Brokopp C D, Seroogy C M, Routes J M. Newborn screening for severe combined immunodeficiency; the Wisconsin experience (2008-2011). J Clin Immunol. 2012; 32:82-8.
11) Kwan A, Church J A, Cowan M J, Agarwal R, Kapoor N, Kohn D B, Lewis D B, McGhee S A, Moore T B, Stiehm E R, Porteus M, Aznar C P, Currier R, Lorey F, Puck J M. Newborn screening for severe combined immunodeficiency and T-cell lymphopenia in California: results of the first 2 years. J Allergy Clin Immunol. 2013; 132:140-50.
12) Vogel B H, Bonagura V, Weinberg G A, Ballow M, Isabelle J, DiAntonio L, Parker A, Young A, Cunningham-Rundles C, Fong C T, Celestin J, Lehman H, Rubinstein A, Siegel S, Weiner L, Saavedra-Matiz C, Kay D M, Caggana M. Newborn screening for SCID in New York State: experience from the first two years. J Clin Immunol. 2014 April; 34(3):289-303.
13) Puck J M. Laboratory technology for population-based screening for severe combined immunodeficiency in neonates: the winner is T-cell receptor excision circles. J Allergy Clin Immunol. 2012 March; 129(3):607-16.
14) Matern D, Tortorelli S, Oglesbee D, Gavrilov D, Rinaldo P (2007) Reduction of the false-positive rate in newborn screening by implementation of MS/MS-based second-tier tests: The Mayo Clinic experience (2004-2007). J Inherit Metab Dis 30: 585-592.
15) Janzen N, Peter M, Sander S et al (2007) Newborn screening for congenital adrenal hyperplasia: additional steroid profile using liquid chromatography-tandem mass spectrometry. J Clin Endocrinol Metab 92:2581-9.
16) Azzari C, la Marca G, Resti M. Neonatal screening for severe combined immunodeficiency caused by an adenosine deaminase defect: a reliable and inexpensive method using tandem mass spectrometry. J Allergy Clin Immunol. 2011 June; 127(6):1394-9.
17) la Marca G, Giocaliere E, Malvagia S, Funghini S, Ombrone D, Della Bona M L, Canessa C, Lippi F, Romano F, Guerrini R, Resti M, Azzari C. The inclusion of ADA-SCID in expanded newborn screening by tandem mass spectrometry. J Pharm Biomed Anal. 2014 January; 88:201-6.
18) la Marca G, Canessa C, Giocaliere E, Romano F, Duse M, Malvagia S, Lippi F, Funghini S, Bianchi L, Della Bona M L, Valleriani C, Ombrone D, Moriondo M, Villanelli F, Speckmann C, Adams S, Gaspar B H, Hershfield M, Santisteban I, Fairbanks L, Ragusa G, Resti M, de Martino M, Guerrini R, Azzari C. Tandem mass spectrometry, but not T-cell receptor excision circle analysis, identifies newborns with late-onset adenosine deaminase deficiency. J Allergy Clin Immunol. 2013; 131:1604-10.
19) Chantin, C., Bonin, B., Boulieu, R., and Bory, 1996. Liquid-chromatographic study of purine metabolism abnormalities in purine nucleoside phosphorylase deficiency. Clin. Chem. 42:326-328.
20) la Marca, G., Canessa, C., Giocaliere, E., Romano, F., Malvagia, S., Funghini, S., Moriondo, M., Valleriani, C., Lippi, F., Ombrone, D., Della Bona, M. L., Speckmann, C., Borte, S., Brodszki, N., Gennery, A. R., Weinacht, K., Celmeli, F., Pagel, J., de Martino, M., Guerrini, R., Wittkowski, H., Santisteban, I., Bali, P., Ikinciogullari, A., Hershfield, M., Notarangelo, L. D., Resti, M., and Azzari, C. 2014b. Diagnosis of immunodeficiency caused by a purine nucleoside phosphorylase defect by using tandem mass spectrometry on dried blood spots. J. Allergy Clin. Immunol. 134:155-159.

Each of the references cited herein is incorporated by reference in its entirety.

In the preceding description, for purposes of explanation, numerous details are set forth in order to provide a thorough understanding of the embodiments. However, it will be apparent to one skilled in the art that these specific details are not required. The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1.-85. (canceled)

86. A multiplex method of measuring adenosine deaminase (ADA) activity in a sample, comprising:
obtaining first and second portions from a dried blood spot (DBS) obtained from blood of a subject,
adding at least one labelled ADA substrate to the first portion,
combining the first portion and the second portion to form a mixture,
measuring a level of the at least one labelled ADA substrate in the mixture to determine ADA activity, and
measuring a level of at least one additional marker in the mixture,
wherein the steps of measuring are carried out by mass spectrometry.

87. The method of claim 86, wherein the first and second portions are obtained from first and second punches from the DBS.

88. The method of claim 87, wherein the first and second punches are each less than 10 mm² in size.

89. The method of claim 86, wherein the first portion is obtained by water extraction.

90. The method of claim 86, wherein the at least one additional marker comprises an endogenous ADA substrate, which comprises adenosine (Ado), and/or deoxyadenosine (dAdo).

91. The method of claim 86, wherein the at least one labelled ADA substrate comprises ¹³C₁₀, ¹⁵N₅ adenosine and/or ¹⁵N₅ deoxyadenosine.

92. The method of claim 86, wherein said step of measuring the level of the at least one labelled ADA substrate further comprising measuring an internal standard comprising another labelled ADA substrate or analogue thereof distinct from the at least one labelled ADA substrate.

93. The method of claim 92, wherein the internal standard comprises ¹³C₁₀ adenosine.

94. The method of claim 86, wherein the steps of measuring are carried out simultaneously.

95. A method of detecting adenosine deaminase (ADA) activity in a sample, comprising:
obtaining two portions of a sample from a dried blood spot (DBS) from blood of a subject,
adding an ADA inhibitor to one of said two portions,
measuring ADA activity in said two portions, and
detecting whether ADA activity is present from the two measured levels, wherein said step of measuring is carried out by mass spectrometry.

96. The method of claim 95, wherein the two portions are obtained from a punch from the DBS.

97. The method of claim 96, wherein the punch is less than 10 mm² in size.

98. The method of claim 95, wherein the sample is obtained by water extraction of the dried blood spot.

99. The method of claim 95, wherein the ADA inhibitor comprises erythro-9-(2-hydroxy-3-nonyl) adenine (EHNA) or pentostatin.

100. The method of claim 99, wherein the ADA inhibitor is EHNA.

101. The method of claim 95, wherein said step of measuring ADA activity comprises measuring levels of at least one ADA substrate added to each of said two portions prior to said step of measuring.

102. The method of claim 101, wherein the at least one ADA substrate is at least one labelled ADA substrate.

103. The method of claim 101, wherein the at least one ADA substrate is at least one fluorescent ADA substrate.

104. The method of claim 102, wherein the at least one labelled ADA substrate comprises ¹³C₁₀, ¹⁵N₅ adenosine and/or ¹⁵N₅ deoxyadenosine.

105. The method of claim 95, wherein the step of measuring further comprises measuring an internal standard.

106. The method of claim 105, wherein the internal standard is added concurrently with stopping ADA activity.

107. The method of claim 105, wherein the internal standard comprises ¹³C₁₀ adenosine.
